(19) United States
(12) Patent Application Publication
(10) Pub. No.: US 2026/0166193 A1
(43) Pub. Date: Jun. 18, 2026

(54) STEAM STERILIZATION PROCESS CHALLENGE DEVICE

(71) Applicant: American Sterilizer Company, Mentor, OH (US)

(72) Inventor: Harry Bala, South Barrington, IL (US)

(21) Appl. No.: 19/129,065

(22) PCT Filed: Nov. 8, 2023
(86) PCT No.: PCT/US2023/079059

§ 371 Date: May 12, 2025

Related U.S. Application Data
(60) Provisional application No. 63/426,948, filed on Nov. 21, 2022.

(51) Int. Cl. A61L 2/28; A61L 2/07; A61L 103/50; G01N 21/78

(52) U.S. Cl. CPC A61L 2/28; A61L 2/07; G01N 21/78; A61L 2103/50

(57) ABSTRACT

A sterilization process challenge device for verifying the efficacy of a steam sterilization process includes first and second walls joined around their peripheries and a chamber defined therebetween for containing at least one sterilization indicator. The sterilization process challenge device also includes an inlet opening and a fluid flow path connected to the inlet opening to provide fluid communication between the inlet opening and the chamber. The fluid flow path is defined in a peripheral seal portion, wherein the only fluid communication between the chamber and an external environment is provided through the inlet opening and the fluid flow path.

### BACKGROUND

The present invention is directed to a sterilization test device for verifying the efficacy of a steam sterilization process.

The sterilization of medical equipment, towels (for hospital and operating room use), gowns and the like is carried out, for the most part, using steam sterilization equipment and methods. For example, a bundle of towels is placed into a steam sterilizer, a vacuum is drawn in the sterilizer to evacuate the air, and steam is introduced to sterilize the bundle of towels.

Due to the nature of the towels being “bundled” it may be difficult to assure that the innermost regions of the towels have been sufficiently subjected to the steam to assure proper levels of sterilization. Essentially, it is a “challenge” for the steam to be introduced to the innermost parts to effect sterilization.

To this end, “challenge packs” or “challenge kits” have been developed to simulate the bundle and more specifically to simulate the difficulty or resistance in reaching the innermost parts: (1) to effect a vacuum; and (2) to introduce steam sufficient to sterilize the local area. There is in fact an ANSI standard for challenge packs that is based upon a bundle of towels having a specific size.

Presently, there are challenge packs on the market. These include paper stacks (stacked like a deck of cards) with an indicator sheet generally in the middle of the stack that can indicate either that a vacuum has been achieved or that a sufficient amount of steam has reached the indicator pack. Another includes a paper stack with a cut out center with a biological indicator vile in the middle. Still another type of indicator includes a plastic container that has a hole in one end, is packed with a permeable material (such as a towel or absorbent paper sheet) and an indicator at the opposite end.

U.S. Pat. No. 9,017,994, which is assigned to the Applicant of the present application and incorporated herein by reference, discloses a sterilization test pack including at least one channel configured to provide a restricted flow path(s) to biological and chemical indicators, in which the at least one channel is defined by a groove(s) or indentation(s) having a depth, a width, and a length. U.S. Pat. No. 11,147,897, which is also assigned to the Applicant of the present application and incorporated herein by reference, discloses a sterilization process challenge device comprising a filter assembly that includes filter members and gaskets to provide a restrictive fluid flow path into a challenge device chamber. Further, U.S. Pat. No. 7,790,105, which is also assigned to the Applicant of the present application and incorporated herein by reference, discloses a sterilization challenge specimen holder including a holder configured to hold a sterilization indicator, such as a biological indicator, and a plug including grooves configured to provide a restricted flow path(s) to the sterilization indicator.

Test devices to verify adequate air removal from a sterilizer chamber are also available. Air that remains in a sterilizer chamber during a steam sterilization process, which is also referred to as a non-condensable gas (NCG), may shield an item being sterilized from the steam and prevent proper sterilization. Bowie-Dick test devices may be used to confirm adequate air removal from sterilizer chambers. A Bowie-Dick test device may be used to qualify sterilizer performance at installation or move or following a major repair. A Bowie-Dick test device may also be used each day the sterilizer is in operation to verify the performance of a pre-vacuum sterilizer by confirming adequate air removal from the sterilizer chamber. The Association for the Advancement of Medical Instrumentation's (AAMI) guidance ANSI/AAMI ST79 Comprehensive Guide to steam sterilization and sterility assurance in health care facilities provides recommended use of the Bowie-Dick Test.

Accurate and cost effective verification of steam sterilization processes and adequate removal of air for pre-vacuum sterilizers is important for obvious reasons. The present disclosure provides a cost effective steam sterilization process test device and Bowie-Dick test device that closely mimics the challenge to reach the innermost regions of a bundle of towels used in the ANSI/AAMI ST79 for sterilization challenge packs.

### BRIEF SUMMARY

In one aspect, a sterilization process challenge device may include a first wall and a second wall joined around their peripheries including a first peripheral seal portion and a chamber defined between the first wall and the second wall for containing at least one sterilization indicator. The sterilization process challenge device may also include an inlet opening and a fluid flow path connected to the inlet opening configured to provide fluid communication between the inlet opening and the chamber. The fluid flow path may be defined in the first peripheral seal portion, wherein the only fluid communication between the chamber and an external environment may be provided through the inlet opening and the fluid flow path. The at least one sterilization indicator may include a biological indicator and/or a chemical indicator.

In some embodiments, the first and second walls may be formed from a steam resistant and heat sealable material, wherein the first and second walls may be heat sealed together to form the first peripheral seal portion. The first peripheral seal portion may include heat sealed portions and unsealed portions, wherein the inlet opening and the fluid flow path may be defined by the unsealed portions in the first peripheral seal portion. In an embodiment, the first and second walls may be formed from a multilayer aluminum foil material comprising an aluminum foil layer and a heat sealable polymeric coating layer. For example, the first and second walls may be formed from a multilayer aluminum foil material comprising an aluminum foil layer and a polypropylene coating layer.

In an embodiment, the fluid flow path may include a first path, a second path, and a third path, wherein the sterilization process challenge device may be configured allow steam to enter through the inlet opening and flow through the first path and through the second path and then through the third path to enter the chamber. The first path may extend between the inlet opening and the second path to provide a fluid communication therebetween, and the third path may extend between the second path and the chamber to provide a fluid communication therebetween. The fluid flow path may include at least two turns including a first turn at a junction between the first path and the second path and a second turn at a junction between the second path and the third path. The first path may extend generally perpendicular to the second path or at an angle less than or greater than 90°, and the third path may extend generally perpendicular to the second path or at an angle less than or greater than 90°.

In an embodiment, the first path may have a length of about ¼ inches to about 1 inch and a width of about ⅛ inches to about ¼ inches, the second path may have a length of about 3 inches to about 5 inches and a width of about ⅛ inches to about ¼ inches, and the third path may have a length of about ¼ inches to about 1 inch and a width of about ⅛ inches to about ¼ inches. For example, the first path may have a length of about 9/32 inches and a width of about 3/16 inches, the second path may have a length of about 4¼ inches and a width of about 3/16, and the third path may have a length of about 9/32 inches and a width of about 3/16 inches.

In an embodiment, the fluid flow path may be configured to provide a tortuous flow path comprising at least one turn and having a total length of about 4 inches to about 6 inches.

The sterilization process challenge device may be configured to exhibit a fail condition after a sterilization process cycle at 132° F. for 4 minutes and exhibit a pass condition after a sterilization process cycle at 132° F. for 10 minutes cycle. The fail condition may include a positive biological indicator result, which indicates at least some microorganisms in the biological indicator survived the sterilization process cycle at 132° F. for 4 minutes, and/or a fail chemical indicator result indicating that the chemical indicator did not reach an endpoint color change after the sterilization process cycle at 132° F. for 4 minutes. The pass condition may include a negative biological indicator result, which indicates all microorganisms in the biological indicator were killed during the sterilization process cycle at 132° F. for 10 minutes, and/or a pass chemical indicator result indicating that the chemical indicator reached an endpoint color change after the sterilization process cycle at 132° F. for 10 minutes.

The sterilization process challenge device may be configured to exhibit a fail condition after a sterilization process cycle at 132° F. for 2 minutes and exhibit a pass condition after a sterilization process cycle at 132° F. for 4 minutes cycle. The fail condition may include a positive biological indicator result, which indicates at least some microorganisms in the biological indicator survived the sterilization process cycle at 132° F. for 2 minutes, and/or a fail chemical indicator result indicating that the chemical indicator did not reach an endpoint color change after the sterilization process cycle at 132° F. for 2 minutes. The pass condition may include a negative biological indicator result, which indicates all microorganisms in the biological indicator were killed during the sterilization process cycle at 132° F. for 4 minutes, and/or a pass chemical indicator result indicating that the chemical indicator reached an endpoint color change after the sterilization process cycle at 132° F. for 4 minutes.

In an embodiment, the sterilization process challenge device may be configured to verify adequate air removal during a sterilization cycle of a pre-vacuum type sterilizer. The sterilization process challenge device may be configured to simulate test methods provided in ANSI/AAMI/ISO 11140-4:2007 or 11140-5:2007 without using the towel test packs specified in the test methods to produce test results that are equivalent to the results obtained using the towel test packs to indicate whether air was adequately removed from the sterilizer during the sterilization cycle. In such an embodiment, the at least one sterilization indicator may be a Class 2 test strip.

In an embodiment, the sterilization process challenge device may be configured to simulate the test described in ANSI/AAAMI/ISO 11140-4:2007 to indicate whether air was adequately removed during a sterilization cycle at 134° C. for 3.5 min±5 sec. In another embodiment, the sterilization process challenge device may be configured to simulate the test described in ANSI/AAAMI/ISO 11140-4:2007 to indicate whether air was adequately removed during a sterilization cycle at 121° C. for 15 min±5 sec. In yet another embodiment, the sterilization process challenge device may be configured to simulate the test described in ANSI/AAAMI/ISO 11140-4:2007 to indicate whether air was adequately removed during a sterilization cycle at 132° C. or 134° C. for 3.5 min±5 sec.

Other aspects, objectives and advantages will become more apparent from the following detailed description.

### BRIEF DESCRIPTION OF THE SEVERAL VIEWS OF THE DRAWINGS

The benefits and advantages of the present invention will become more readily apparent to those of ordinary skill in the relevant art after reviewing the following detailed description and accompanying drawings, wherein:

FIG. 1 is a schematic illustration of a sterilization process challenge device according to an embodiment;

FIG. 2 is a cross sectional view of the sterilization process challenge device of FIG. 1 cut across A-A;

FIG. 3 is a schematic illustration of a heat sealing device according to an embodiment;

FIG. 4 is a schematic illustration of a sterilization process challenge device including a tortuous fluid flow path according to an embodiment; and

FIG. 5 is a picture of a sterilization process challenge device formed from a heat sealable aluminum foil material according to an embodiment.

For simplicity and clarity of illustration, elements shown in the figures may not be drawn to scale. For example, the dimension of some of the elements may be exaggerated relative to each other for clarity.

### DETAILED DESCRIPTION

While the present disclosure is susceptible of embodiment in various forms, there will hereinafter be described presently preferred embodiments with the understanding that the present disclosure is to be considered an exemplification and is not intended to limit the disclosure to the specific embodiments illustrated.

It should be further understood that the title of this section of this specification, namely, “Detailed Description”, relates to a requirement of the United States Patent Office, and does not imply, nor should be inferred to limit the subject matter disclosed herein.

A sterilization process challenge device (“PCD”) according to various embodiments is provided. The PCD may be configured to hold a biological indicator and/or a chemical indicator to test the efficacy of a steam sterilization process and/or verify adequate removal of air from a sterilizer chamber. For example, the PCD may be used to verify the efficacy of a steam sterilization process or used as a Bowie-Dick test device to evaluate the performance of a pre-vacuum sterilizer by confirming adequate air removal from the sterilizer chamber. The biological indicator may contain microorganisms, such as Escherichia coli, Legionella sp., Campylobacter sp., Staphylococcus, Streptococcus species and Cryptosporidium.

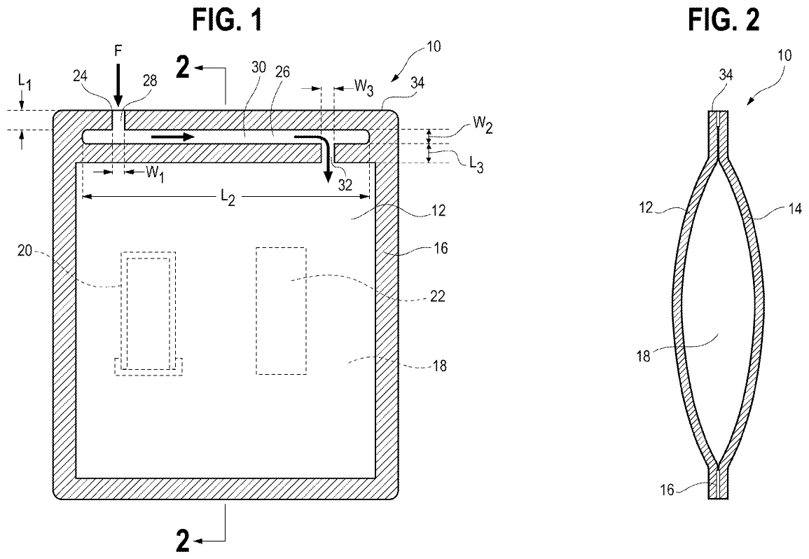

FIGS. 1 and 2 illustrate a PCD 10 according to an embodiment. The PCD 10 may generally comprise a first wall 12 and a second wall 14 joined around their peripheries with a chamber 18 defined therebetween and an inlet opening 24. The chamber 18 may be configured to contain a biological indicator 20 and/or a chemical indicator 22. The PCD 10 may be formed from a suitable steam resistant material and closed around the peripheries except the inlet opening 24, such that an only fluid flow path from an external environment into the chamber 18 is provided through the inlet opening 24. In the embodiment of FIG. 1, the PCD 10 may have a generally rectangular shape. In other embodiments, the PCD 10 may be provided in various shapes, such as a circular, oval, etc.

The PCD 10 may include a fluid flow path 26 for steam to enter the chamber 18. The fluid flow path 26 may be configured to provide a flow restriction that closely mimics the challenge for steam to reach the innermost regions of the bundle of towels used in the ANSI/AAMI ST79 for sterilization challenge packs. In an embodiment, the first and second walls 12, 14 may be sealed together around their peripheries, for example, via heat sealing, to form a peripheral seal 16. In such an embodiment, the fluid flow path 26 may be defined by unsealed areas in the peripheral seal 16. For example, a heat sealing device configured to provide predetermined unsealed areas may be used to seal the peripheries of the PCD 10 to form the fluid flow path 26 in the peripheral seal 16.

In the embodiment of FIG. 1, the fluid flow path 26 may be defined by the unsealed areas in the peripheral seal 16 and may include a first path 28, a second path 30, and a third path 32. The PCD 10 may be configured such that steam may enter through the inlet opening 24 and flow through the first path 28 and through the second path 30 and then through the third path 32 to enter the chamber 18.

In an embodiment, the second path 30 may be configured to extend generally parallel to a first peripheral edge 34 of the PCD 10 as shown in FIG. 1. The first path 28 may extend between the inlet opening 24 and the second path 30 to provide fluid communication therebetween. The first path 28 may extend generally perpendicular to the second path 30 as shown in FIG. 1 or at an angle less than or greater than 90° to provide a turn in the fluid flow path 26. The third path 32 may extend between the second path 30 and the chamber 18 to provide fluid communication therebetween. The third path 32 may extend generally perpendicular to the second path 30 as shown in FIG. 1 or at an angle less than or greater than 90° to provide a turn in the fluid flow path 26. In some embodiments, the second path 30 may extend slanted having a non-zero slope relative to the first peripheral edge 34. In the embodiment of FIG. 1, the fluid flow path 26 may be configured such that steam entering through the inlet opening 24, as shown with an arrow F, may flow through the fluid flow path 26 taking turns at the junction of the first path 28 and the second path 30 and at the junction of the second path 30 and the third path.

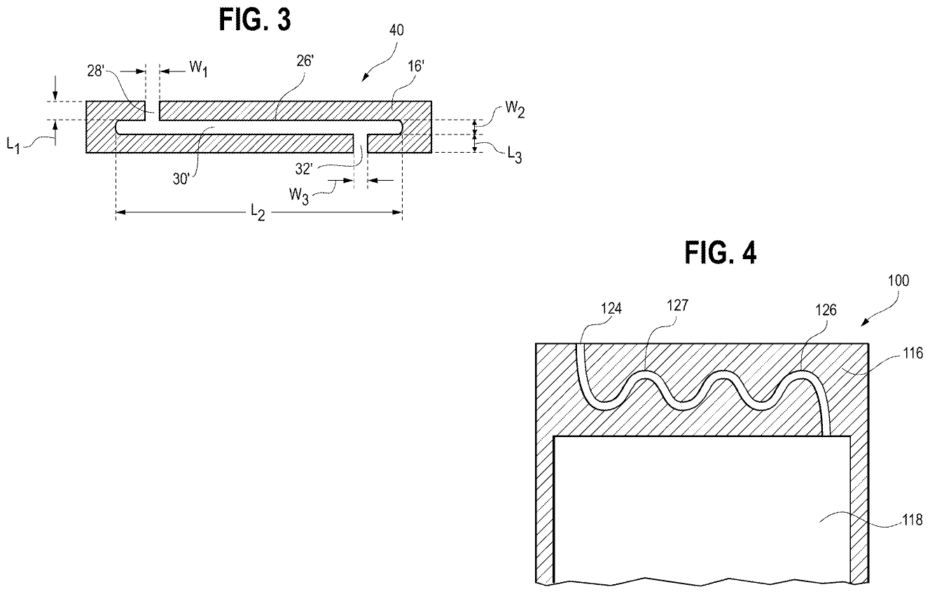

FIG. 3 is a schematic illustration of a heat sealing device 40 according to an embodiment. The heat sealing device 40 may be configured to form a portion of the peripheral seal 16 including the fluid flow path 26. The heat sealing device 40 may generally include a peripheral portion 16′ and an unsealing portion 26′ including a first portion 28′, a second portion 30′, and a third portion 32′. When used to make the PCD 10, the first and second walls 12, 14 adjacent the peripheral portion 16′ may be sealed together to form the peripheral seal 16, while the first and second walls 12, 14 adjacent the unsealing portion 26′ may be left unsealed to form the fluid flow path 26 comprising the first, second, and third paths 28, 30, 32.

The fluid flow path 26 may be configured to have a total length of about 3 inches to about 7 inches, preferably about 4 inches to about 6 inches, wherein the first path 28 may have a length L₁ of about ⅛ inches to about 1½ inches, preferably about ¼ inches to about 1 inch, and a width W₁ of about 1/16 inches to about ½ inches, preferably about ⅛ inches to about ¼ inches, wherein the second path 30 may have a length L₂ of about 2 inches to about 6 inches, preferably about 3 inches to about 5 inches, and a width W₂ of about 1/16 inches to about ½ inches, preferably about ⅛ inches to about ¼ inches, and wherein the third path 32 may have a length L₃ of about ⅛ inches to about 1½ inches, preferably about ¼ inches to about 1 inch, and a width W₃ of about 1/16 inches to about ½ inches, preferably about ⅛ inches to about ¼ inches. In an embodiment, the fluid flow path 26 may be configured to have a total length of about 413/16 inches, wherein the first path 28 may have a length L₁ of about 9/32 inches and a width W₁ of about 3/16 inches, the second path 30 may have a length L₂ of about 4¼ inches and a width W₂ of about 3/16, and the third path 32 may have a length L₃ of about 9/32 inches and a width W₃ of about 3/16 inches.

In some embodiments, the fluid flow path may be configured to provide various tortuous flow paths. For example, FIG. 4 shows a PCD 100 including a fluid flow path 126 defined by unsealed areas in a peripheral seal 116, wherein the fluid flow path 126 may be configured to provide a tortuous flow path including a plurality of turns 127, e.g., a serpentine flow path, to mimic the flow restriction that steam may face to reach the innermost regions of the bundle of towels used in ANSI/AAMI ST79 for verifying sterilization processes.

The first and second walls 12, 14 may be formed from a suitable steam resistant material, such as a multilayer material comprising a metallic foil layer and a polymeric heat sealable layer. In an embodiment, the first and second walls 12, 14 may be formed from a heat sealable aluminum foil material having a thickness of about 0.002 inches to about 0.005 inches and comprising an aluminum foil layer and a heat sealable polymeric coating layer. For example, the first and second walls 12, 14 may be formed from a heat sealable aluminum foil material having a total thickness of about 0.003 inches and comprising and aluminum foil layer and a heat sealable polypropylene coating layer, wherein the aluminum foil layer has a thickness of about 0.002 inches and a heat sealable polypropylene coating layer has a thickness of about 0.001 inches, such as FastelFoil PRO™ available from Fastel Adhesive & Substrate Products.

In an embodiment, the PCD 10, 100 may be formed from a heat sealable aluminum foil material, wherein the chamber 18 may be sized and configured to hold a biological indicator 20 and/or a chemical indicator 22. For example, the chamber 18 may contain a biological indicator 20, such as a self-contained biological indicator (SCBI).

In use, the PCD 10, 100 may be placed in a sterilizer chamber along with objects to be sterilized. During a sterilization process cycle, steam may flow into the chamber 18, 118 through the inlet opening 24, 124 and through the fluid flow path 26, 126, wherein the flow of steam is restricted by the configuration of the fluid flow path 26, 126. At the end of the sterilization process cycle, a user may cut open the PCD 10, 100 to remove the biological indicator 20 and/or chemical indicator 22 to evaluate the efficacy of the sterilization process. The PCD 10, 100 may be configured such that the flow of steam may be restricted by the configuration of the fluid flow path 26, 126 to closely mimic the flow resistance into the center of 16-towel test pack used in the ANSI/AAMI ST79 for sterilization challenge packs.

In some sterilization processes, a vacuum may be drawn in a sterilizer chamber, following which steam is introduced into the chamber. In such sterilization processes, the PCD 10, 100 may be used to verify adequate air removal from the sterilizer chamber. In an embodiment, the PCD 10, 100 may be configured to verify adequate air removal during a sterilization cycle of a pre-vacuum type sterilizer. The PCD 10, 100 may be configured to simulate the test methods provided in ANSI/AAMI/ISO 11140-4:2007 and 11140-5:2007 without using the towel test packs specified in these test methods. The PCD 10, 100 may include a test strip, such as a Class 2 test strip, in the chamber 18, 118, and configured specific to the test method and sterilization cycle parameters to produce test results that are substantially equivalent to that produced using the test packs described in the test method. For example, ANSI/AAMI/ISO 11140-5:2007 requires that an indicator system when placed in the center of a specified test pack should show a non-uniform color change when the temperature at the center of the test pack is 2° C. lower than the temperature of the chamber drain at the beginning of the final 1 min of a 3.5 min cycle at 134° C., or at the beginning of the final 5 min of a 15 min cycle at 121° C. of the exposure phase of a sterilizer to indicate inadequate air removal. The PCD 10, 100 may be configured to simulate this test to produce test results that are substantially equivalent to the results obtained using the test pack and indicator system described in the test method to indicate whether air was adequately removed from a sterilizer during a sterilization cycle.

In an embodiment, the PCD 10, 100 including a Class 2 test strip may be configured to simulate a test described in ANSI/AAAMI/ISO 11140-4:2007 to indicate whether air was adequately removed during a sterilization cycle at 134° C. for 3.5 min±5 sec. In another embodiment, the PCD 10, 100 including a Class 2 test strip may be configured to simulate a test described in ANSI/AAMI/ISO 11140-4:2007 to indicate whether air was adequately removed during a sterilization cycle at 121° C. for 15 min±5 sec. In yet another embodiment, the PCD 10, 100 including a Class 2 test strip may be configured to simulate a test described in ANSI/AAMI/ISO 11140-5:2007 to indicate whether air was adequately removed during a sterilization cycle at 132° C. or 134° C. for 3.5 min±5 sec.

In an embodiment, a method of verifying adequate air removal during a sterilization cycle of a pre-vacuum type sterilizer may include the steps of placing the PCD 10, 100 including a Class 2 test strip in a pre-vacuum type sterilizer, running a pre-vacuum sterilization cycle, and verifying adequate air removal by inspecting a color change of the Class 2 test strip, the color change of the Class 2 test strip being in response to adequate removal of air from the PCD 10, 100 after exposure to saturated steam, wherein the method simulates a test described in ANSI/AAMI/ISO 11140-4:2007 to indicate whether air was adequately removed during a sterilization cycle at 134° C. for 3.5 min±5 sec or a sterilization cycle at 121° C. for 15 min±5 sec or a sterilization cycle at 132° C. or 134° C. for 3.5 min±5 sec.

Some prior art challenge packs include relatively complex assemblies and/or components, such as an absorber or other sterilant-reactive device, to restrict the flow of steam or gaseous sterilization medium, which may require relatively complex manufacturing steps and/or relatively expensive materials. The PCD according to various embodiments of the present disclosure is relatively simple and cost effective to make and may be configured to provide the restrictive flow path that closely mimics that of the 16-towel pack. As such, the PCD of the present disclosure may be provided as a cost effective disposable sterilization process challenge device.

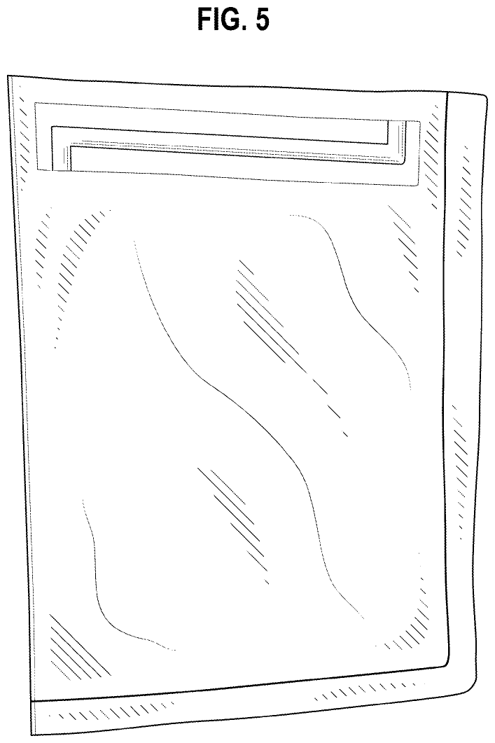

Samples of the PCD of FIGS. 1 and 2 were prepared and tested to evaluate the performance of the PCD. The PCD samples were formed from a heat sealable aluminum foil material comprising an aluminum foil layer having a thickness of about 0.002 inches and a heat sealable polypropylene coating layer having a thickness of about 0.001 inches. The PCD samples included the fluid flow path 26 comprising the first, second, and third paths 28, 30, 32, wherein the first path 28 had a length L₁ of about 9/32 inches and a width W₁ of about 3/16 inches, the second path 30 had a length L₂ of about 4¼ inches and a width W₂ of about 3/16, and the third path 32 had a length L₃ of about 9/32 inches and a width W₃ of about 3/16 inches FIG. 5 shows a picture of the PCD sample.

The PCD samples were place in a steam sterilization chamber for a sterilization process cycle at 132° F. for 4 minutes (min), 5 min, 6 min, 8 min, or 10 min and evaluated for sterilization process challenge characteristics. The PCD samples that exhibit fail conditions after a sterilization process cycle at a 132° F. for 4 minutes and pass conditions after a sterilization process cycle at a 132° F. for 10 minutes were considered to provide a sterilization process challenge comparable to the restrictive flow path into the center of 16-towel test pack used in the ANSI/AAMI ST79 for sterilization challenge packs. The fail conditions included a positive biological indicator result indicating that at least some microorganisms in the biological indicator survived the steam sterilization process cycle and/or a fail chemical indicator result indicating that the chemical indicator did not reach endpoint color changes. The pass conditions included a negative biological indictor result indicating that all microorganisms were killed during a steam sterilization process cycle and/or a pass chemical indicator result indicating that the chemical indicator reached the endpoint color changes. The test results showed that the PCD samples exhibited fail conditions after a sterilization process cycle at 132° F. for 4 minutes while exhibiting pass conditions after a sterilization process cycle at a 132° F. for 10 minutes to indicate that the PCD samples provided a sufficient flow resistance to mimic the flow resistance to reach the center of the 16-towel pack.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

1. A sterilization process challenge device, comprising:
a first wall and a second wall joined around their peripheries including a first peripheral seal portion;
a chamber defined between the first wall and the second wall configured to contain at least one sterilization indicator;
an inlet opening; and
a fluid flow path connected to the inlet opening configured to provide a fluid communication between the inlet opening and the chamber, the fluid flow path defined in the first peripheral seal portion;
wherein the only fluid communication between the chamber and an external environment is provided through the inlet opening and through the fluid flow path.

2. The sterilization process challenge device of claim 1, wherein the first and second walls are formed from a steam resistant and heat sealable material, wherein the first and second walls are heat sealed together to form the first peripheral seal portion, wherein the first peripheral seal portion includes heat sealed portions and unsealed portions, wherein the inlet opening and the fluid flow path are defined by the unsealed portions in the first peripheral seal portion.

3. The sterilization process challenge device of claim 1, wherein the first and second walls are formed from a multilayer aluminum foil material comprising an aluminum foil layer and a heat sealable polymeric coating layer.

4. The sterilization process challenge device of claim 1, wherein the first and second walls are formed from a multilayer aluminum foil material comprising an aluminum foil layer and a polypropylene coating layer.

5. The sterilization process challenge device of claim 1, wherein the fluid flow path is configured to provide a tortuous flow path comprising at least one turn.

6. The sterilization process challenge device of claim 1, wherein the fluid flow path has a total length of about 4 inches to about 6 inches.

7. The sterilization process challenge device of claim 1, wherein the fluid flow path includes a first path, a second path, and a third path, wherein the sterilization process challenge device is configured to allow steam to enter through the inlet opening and flow through the first path and through the second path and then through the third path to enter the chamber.

8. The sterilization process challenge device of claim 7, wherein the first path extends between the inlet opening and the second path to provide a fluid communication therebetween, and the third path extends between the second path and the chamber to provide a fluid communication therebetween, wherein the fluid flow path includes at least two turns including a first turn and a second turn, wherein the first turn is provided at a junction between the first path and the second path, and the second turn is provided at a junction between the second path and the third path.

9. The sterilization process challenge device of claim 8, wherein the first path extends generally perpendicular to the second path, and the third path extends generally perpendicular to the second path.

10. The sterilization process challenge device of claim 8, wherein the first path extends to the second path at an angle less than or greater than 90°, and the third path extends from the second path at an angle less than or greater than 90°.

11. The sterilization process challenge device of claim 7, wherein the first path has a length of about ¼ inches to about 1 inch and a width of about ⅛ inches to about ¼ inches, the second path has a length of about 3 inches to about 5 inches and a width of about ⅛ inches to about ¼ inches, and the third path has a length of about ¼ inches to about 1 inch and a width of about ⅛ inches to about ¼ inches.

12. The sterilization process challenge device of claim 1, wherein the first path has a length of about 9/32 inches and a width of about 3/16 inches, the second path has a length of about 4¼ inches and a width of about 3/16, and the third path has a length of about 9/32 inches and a width of about 3/16 inches.

13. The sterilization process challenge device of claim 1, wherein the at least one sterilization indicator includes a biological indicator and/or a chemical indicator.

14. The sterilization process challenge device of claim 1, wherein the sterilization process challenge device is configured to exhibit a fail condition after a sterilization process cycle at 132° C. for 4 minutes and exhibit a pass condition after a sterilization process cycle at 132° C. for 10 minutes cycle, wherein the fail condition includes a positive biological indicator result indicating at least some microorganisms in the biological indicator survived the sterilization process cycle at 132° C. for 4 minutes and/or a fail chemical indicator result indicating that the chemical indicator did not reach an endpoint color change after the sterilization process cycle at 132° C. for 4 minutes, and wherein the pass condition includes a negative biological indicator result indicating all microorganisms in the biological indicator were killed during the sterilization process cycle at 132° C. for 10 minutes and/or a pass chemical indicator result indicating that the chemical indicator reached an endpoint color change after the sterilization process cycle at 132° C. for 10 minutes.

15. The sterilization process challenge device of claim 1, wherein the sterilization process challenge device is configured to exhibit a fail condition after a sterilization process cycle at 132° C. for 2 minutes and exhibit a pass condition after a sterilization process cycle at 132° C. for 4 minutes cycle, wherein the fail condition includes a positive biological indicator result indicating at least some microorganisms in the biological indicator survived the sterilization process cycle at 132° C. for 2 minutes and/or a fail chemical indicator result indicating that the chemical indicator did not reach an endpoint color change after the sterilization process cycle at 132° C. for 2 minutes, and wherein the pass condition includes a negative biological indicator result indicating all microorganisms in the biological indicator were killed during the sterilization process cycle at 132° C. for 4 minutes and/or a pass chemical indicator result indicating that the chemical indicator reached an endpoint color change after the sterilization process cycle at 132° C. for 4 minutes.

16. The sterilization process challenge device of claim 1, wherein the sterilization process challenge device is configured to verify adequate air removal during a sterilization cycle of a pre-vacuum type sterilizer, wherein the sterilization process challenge device is configured to simulate test methods provided in ANSI/AAMI/ISO 11140-4:2007 or 11140-5:2007 without using towel test packs specified in the test methods to produce test results that are equivalent to results obtained using the towel test packs to indicate whether air was adequately removed from the sterilizer during the sterilization cycle.

17. The sterilization process challenge device of claim 16, wherein the sterilization process challenge device is configured to simulate a test described in ANSI/AAAMI/ISO 11140-4:2007 to indicate whether air was adequately removed during a sterilization cycle at 134° C. for 3.5 min±5 sec.

18. The sterilization process challenge device of claim 16, wherein the sterilization process challenge device is configured to simulate a test described in ANSI/AAAMI/ISO 11140-4:2007 to indicate whether air was adequately removed during a sterilization cycle at 121° C. for 15 min±5 sec.

19. The sterilization process challenge device of claim 16, wherein the sterilization process challenge device is configured to simulate a test described in ANSI/AAAMI/ISO 11140-4:2007 to indicate whether air was adequately removed during a sterilization cycle at 132° C. or 134° C. for 3.5 min±5 sec.

20. The sterilization process challenge device of claim 16, wherein the at least one sterilization indicator is a Class 2 test strip.
